(19) United States
(12) Patent Application Publication
(10) Pub. No.: US 2005/0004468 A1
(43) Pub. Date: Jan. 6, 2005

(54) Increased sensitivity for 4-D ultrasound imaging and for 4-D doppler ultrasound imaging

(75) Inventors: Kenneth Abend, Huntingdon Valley, PA (US); Christopher X Miller, Audubon, PA (US)

Correspondence Address: John K. Kim, Esq.;McCarter & English, LLP, Four Gateway Center, 100 Mulberry Street, Newark, NJ, 07102, US

(73) Assignee: VUESONIX SENSORS, INC.

(21) Appl. No.: 10/801,495

(22) Filed: Mar. 16, 2004

Related U.S. Application Data
(60) Provisional application No. 60/455,431, filed on Mar. 17, 2003.

(51) Int. Cl. A61B 8/06

(52) U.S. Cl. 600/453

(57) ABSTRACT

For real-time Three Dimensional ultrasonic Doppler imaging, a number of techniques are combined in order to increase detection sensitivity. This disclosure is directed, primarily, to improved filter design employing transient removal, mean removal and/or trend removal.

### CROSS-REFERENCE TO RELATED APPLICATION

This application claims the benefit of U.S. Provisional Patent Application Ser. No. 60/455,431, filed Mar. 17, 2003, the disclosure of which is incorporated herein by reference in its entirety.

### FIELD OF THE INVENTION

The invention is in the field of ultrasound imaging, primarily for medical purposes.

### BACKGROUND OF THE INVENTION

Several methods have recently been invented for real-time three-dimensional (3-D) ultrasound imaging, often referred to as 4-D imaging, the 4^{th} dimension being time. Attempts at 4-D imaging often result in a decrease in sensitivity. This is because a 2-D imaging system that requires Nbeams (often called lines) implies a 3-D volume that requires N² beams (lines), if each plane of the volume is to have the same resolution as the 2-D system. Producing all these lines at a specified frame rate means either (1) dwelling at each position for a shorter duration or (2) using a broad transmit beam and forming several receive beams at a time. Either approach implies a potential loss of gain. Method 1, as described in U.S. Pat. No. 6,238,346, implies a slower frame rate and/or a small number of pulses per dwell. The latter is particularly disastrous for Doppler imaging (Power or Color) because of the pulses required by the wall filter to detect the Doppler signal. Method 2, as described in U.S. Pat. No. 6,524,253 (the '253 patent) and U.S. Pat. No. 6,682,483 (the '483 patent), spreads the transmitted energy to cover multiple beam positions. This lowering of energy density results in lower sensitivity. This is partially compensated for by dynamically focusing the receive array in two cross-range directions. The disclosures of U.S. Pat. No. 6,524,253 and U.S. Pat. No. 6,682,483 are hereby incorporated by reference in their entirety herein.

### SUMMARY OF THE INVENTION

A number of approaches have been developed to maximize the overall signal to noise ratio and hence increase sensitivity. In addition, approaches/methods have been developed, in particular, to improve Doppler sensitivity, and hence improve the detection of blood flow. These methods, described below, may be employed singularly or in combination. While some of the methods described below are known to those skilled in the art, it is the combination of several such methods that achieves the sensitivity enhancement needed for 4-D Ultrasound Imaging.

Several methods are briefly described in the sections below and some are more fully described in the documents incorporated herein by reference above. The use of narrowband signals and long pulses significantly increases the average signal power and reduces the noise. The use of only a small portion of the array at a time for transmission results in a significant reduction in transmitter duty cycle and hence time-averaged heating, which normally limits transmitter power. The transmit beam is shaped to concentrate power in the region spanned by the collection of receive beams and the high pass wall filter is designed to take advantage of the increased number of pulses available. A new method for Power Doppler calculation is used to significantly improve the signal-to-noise ratio. The increased number of pulses allows for detection of very slow moving blood, the increased frame rate provides for integration gain, and the lowered transmitter duty cycle allows for the use of coded waveforms to increase the duty cycle and hence provide greater average power without decreasing the range resolution. Coherent processing gain is maintained over long pulse-Doppler dwells and various methods are used for making efficient use of the pulses. Such methods include the interleaving of pulse trains, and the utilization of mean and/or trend removal in place of or in addition to other means of high-pass Doppler wall filtering. The disclosure and claims of this application are directed, primarily, to an improved filter design, incorporating mean removal and/or trend removal.

Further features and advantages of the invention will appear more clearly on a reading of the following detailed description of exemplary embodiments of the invention, which are given below by way of example only, with reference to the accompanying drawings.

### BRIEF DESCRIPTION OF THE DRAWINGS

FIG. 1 is a schematic representation of an exemplary system data flow.

FIGS. 2A-D are schematic illustrations of the spectral positions of exemplary data streams at four digital processing states.

FIG. 3 is a schematic depiction of an exemplary processing signal flow in a system employing a wall filter.

FIG. 4 is a schematic depiction of an exemplary processing signal flow in a system employing a wall filter and a mean removal filter.

FIG. 5 is a schematic depiction of an exemplary processing signal flow in a system employing a mean removal filter and a trend removal filter.

FIG. 6 is a schematic depiction of an exemplary processing signal flow in a system employing a wall filter and a mean removal filter and trend removal filter.

FIG. 7 a schematic depiction of an exemplary processing signal flow in a system employing a finite impulse response (FIR) filter with transient elimination.

### DETAILED DESCRIPTION OF THE INVENTION

Analog and Digital Data Flow

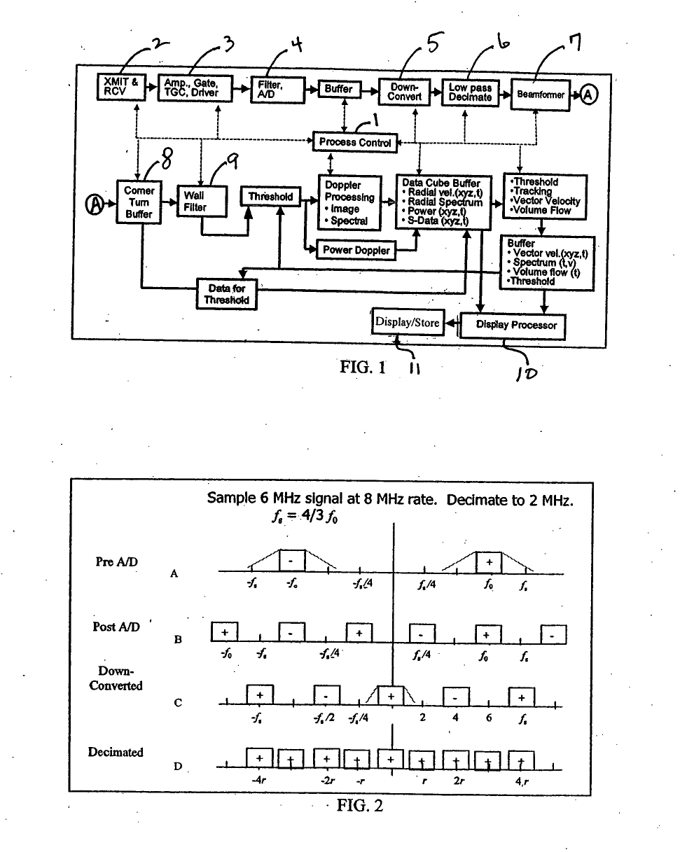

FIG. 1 schematically illustrates the analog and digital data flow in an exemplary system implementing the system generally disclosed in the '483 patent. The process control 1 controls the timing and phasing of the signals 2 transmitted by the probe's preselective transmitting and receiving elements. The received signals are amplified 3, filtered and digitized 4, down converted 5 to place all unshifted signals at DC, and decimated 6 for efficient processing. The beamformer 7, under control of the process control 1, analytically progressively phase adjusts the input data to look at a given direction and depth (range).

This information is buffered 8, digitally filtered 9 and further digitally processed to produce the signals 10 to be recorded or displayed 11. The signals 10 can include vector velocity and flow volume. The digital processing is performed either in individual, task-specific processors, such as Field Programmable Gate Arrays, or in general purpose computers under software control.

Narrow-Band Probe for Increased Sensitivity

Because of the general manufacturing approach of ultrasound transducers their sensitivity is generally inversely proportional to the bandwidth of the transducer. The noise (not including speckle) of an ultrasound system is generally a function of the preamplifier (thermal noise from the real load presented to the transducer and the junction noise of the amplifier) and its bandwidth. Thus all other things being equal the signal to noise ratio will be inversely proportional to the bandwidth of the transducer and the preamplifier. In order to optimize the signal to noise ratio, thus, the useful depth that the ultrasound system can operate, the transducer bandwidth must be matched to the pulse width of the system. For example, for pulse Doppler operation using a pulse width of 1 microsecond, the bandwidth of the transducer should be approximately 1 divided by 1 microsecond or 1 megahertz. Thus the transducer should be optimized for a 1 megahertz bandwidth. The receiver processing bandwidth should also be 1 megahertz.

Band Pass Sampling for Narrowband Processing

In any digitally beamformed ultrasound system used for 3-D or 4-D imaging, the number of piezoelectric elements and analog to digital (A/D) converters is large. To keep the input data rate manageable, the elements are sampled at a low Nyquist rate, determined by the signal bandwidth, instead of at the usual Nyquist rate that exceeds twice the operating frequency. In one particular implementation, illustrated in FIG. 2, the carrier frequency is f₀=6 MHz but the AID sampling rate is 8 MHz instead of being greater than 12 MHz. The illustrated 8 MHz real sampling rate allows for computationally efficient decimation to complex sample rates of 1, 2, or 4 MHz, to match the bandwidth of the transmitted signal. An anti-aliasing bandpass filter precedes the A/D converter. This restriction of the bandwidth before the A/D converter, followed by more detailed filtering in the digital sample-rate decimation filters, limits the noise at the system input to the narrow signal bandwidth and therefore optimizes the signal-to noise ratio (SNR).

In FIG. 2 line A represents the spectrum of the analog signal received at a typical transducer element, where the dotted lines represent the frequency response of a band-pass anti-aliasing filter. Line B represents the spectrum of the signal in A after it has been band-pass filtered and sampled at the rate f_{S}(=8 MHz). The spectrum is periodic (in the frequency domain) with period f_{S}. Line C is the result of digitally heterodyning the signal in B, multiplying it by exp{j2π(f_{S}/4)t}. Since t=n/f_{S}, the multiplier is simply exp(jπn/2)=jⁿ. The dotted lines represent the frequency response of a digital low-pass decimation filter. Line D shows the spectrum of the signal after it is decimated by a factor of four by computing only every fourth sample at the output of the decimation filter. The new sampling rate (for this particular example) is r=f_{S}/4=2 MHz

Reduced Heating Via Distributed Transmission

In conventional ultrasound designs using a one-dimensional array the transmitter power is limited by two factors, heat on the probe surface and/or sound pressure. Its linear array has limited surface area to dissipate the heat generated by the elements and there is a large overlap in active elements as the transmitting subarray moves along the linear array. The duty cycle of any transmit element is quite high. The transmitter is focused (increasing power density) to produce high sound pressure levels at a focal point within the imaging volume. The transmit power is limited to a level such that the high pressure at the focal point produces a mechanical index number (MI) that is below the FDA limit set by the US Food and Drug Administration (FDA).

The new design has a two-dimensional array and uses a transmitter that utilizes N by M elements. The transmitter is shaped to produce an insonification column (i.e., a column of ultrasonic energy) of unchanging or decreasing power density and thus can utilize a higher power without exceeding the MI at any one point i.e. a focus point. The result is insonification everywhere in the column can be equal to what is normally only achieved at the focus of a conventional design. If the new design transmitter were stationary with this higher power there would be heating concerns that would limit the power that could be used. As part of the design the activated group of transmitting elements is moved around the two dimensional array, spreading the heat and keeping it within FDA limits. The duty cycle of a transmitting element can be quite low.

Shaping of the Transmit Beam

In the '483 patent, entitled “Transmitter Patterns for Multibeam Reception,” the transmitter pattern is shaped to have constant gain in the region of the desired receiver beams while greatly attenuating the receiver grating lobes. The transmit pattern is also designed to drop off sharply outside of the region of constant gain. This helps minimize the loss of transmit gain so that the amount of transmitter energy density that is sacrificed in exchange for increased signal duration is minimized.

Improved Wall Filter Performance

The availability of a large number of pulses due to the simultaneous formation of multiple beams is taken advantage of in a flexible wall filter digitally implemented in a Field Programmable Gate Array.

Autocorrelation Amplitude for Power Doppler

This method for improving the signal-to-noise ratio is described in U.S. application Ser. No. 10/764,657, filed Jan. 26, 2004, the disclosure of which is incorporated herein by reference in its entirety. Power Doppler is obtained from multiple pulses, not by averaging their powers, but by computing the autocorrelation function at a lag of one transmit pulse. It is well known that the angle (or argument) of this complex autocorrelation is an extremely robust and accurate estimate of velocity for use in Color Doppler Imaging. (See, for example, Chihiro Kasai, Koroku Namekawa, Akira Koyano, and Ryozo Omoto, “Real-Time Two-Dimensional Blood Flow Imaging Using an Autocorrelation Technique”, IEEE Transactions on Sonics and Ultrasonics, Vol. SU-32, No. 3, May 1985). What is the subject of the above-cited application is that the amplitude of this very same complex autocorrelation is an extremely robust and accurate estimate of the flow power for Power Doppler Imaging, and is far less noisy than the value obtained by averaging the power at the output of the wall filter.

Doppler Gain from Increased Number of Pulses

By creating simultaneous receiver beams, the number of pulses per beam used to establish the Doppler measurement is increased. Since the transmitter must insonify several beams, transmit gain (or power density) is sacrificed in exchange for time or number of pulses. (See the '253 and '483 patents.) The additional pulses provide signal-processing gain. The autocorrelation method of the preceding paragraph provides the equivalent of coherent integration when measuring Doppler signals. In a Doppler Ultrasound system, the additional pulses do double duty. They provide signal-processing gain, while they also provide enhanced Doppler resolution to improve the detectability of slow moving blood.

The benefit of increased number of pulses in a Doppler system is highly non-linear. Given, for example, 24 pulses per receiver beam, we can sacrifice, for example, 8 pulses by using a 9-tap Finite Impulse Response high-pass wall filter and eliminating the initial (as well as the final) 8 pulses from the output. In this manner, stationary clutter is completely removed (infinitely attenuated) by simply using a filter whose tap weights sum to zero. In this example, the 24 input pulses result in 24=8=16 output pulses that are devoid of clutter. If no more than 8 input pulses were available, a 9-tap wall filter would not be usable in the manner described. If a 7-tap filter were used, only 2 pulses would remain out of 8 while 18 would remain out of 24. This produces an SNR improvement of 10 log (18/2)=9.5 dB by tripling the number of pulses. Slower moving blood requires longer wall filters in order to provide greater SNR improvement.

Frame Rate Enhancement

The forming of simultaneous receive beams for each transmit pulse (See the '253 and '483 patents.) completely covers the volume of interest in far less time than a conventional single beam system, even if it is electronically steered. The increased frame rate allows integration to provide an additional SNR improvement as well as permits the use of more pulses for a given frame rate, which permits the other methods to be employed.

Spread Spectrum

The pulse repetition frequency (PRF) is usually chosen low enough to avoid range ambiguities and high enough to prevent Doppler ambiguities (aliasing). A wide bandwidth, for fine range resolution, is usually achieved by use of a short pulse. This generally provides a low duty cycle. However, if SNR is a problem, fine range resolution can be achieved by compressing a longer phase coded pulse. The longer pulse provides more average power for a specified peak power. The over-all average power at a given point on the probe is limited because only a small section of the probe is active at any given time. Thus, over the duration of a complete frame, the duty cycle is low because only a small portion of the probe transmits at any instant. This allows a higher than usual peak power without heating the probe. When peak power is limited, using a longer coded waveform can increase the duty factor. Any of a large number of spread spectrum techniques could be utilized. An exemplary implementation employs a Barker code.

Interleaving for Efficient Pulse Repetition Frequency Reduction

The concept of interleaving for ultrasound Doppler gain is introduced in U.S. application Ser. No. 10/764,658, filed Jan. 26, 2004, the disclosure of which is incorporated herein by reference in its entirety. The concept is to interleave pulses so as to lower the effective pulse repetition frequency (PRF) in order to image low blood velocities, while still utilizing the total number of pulses obtained at the higher PRF, for improved SNR.

Mean Removal and Trend Removal

The high-pass wall filter in FIG. 1 is difficult to design without requiring use of a large number of pulses collected over a long duration. Normal time-invariant linear filters involve transients and a consequent loss of data if the transient data is deleted. However, deletion of transient data can lead to a filtering algorithm that removes the BMODE (unshifted) component in the data stream.

Removing (subtracting) the mean and/or a trend attenuates low frequencies without any loss of data. The herein-disclosed wall filter is designed to operate with various combinations of filtering, mean removal, and trend removal. This is discussed in subsections a. through c. below.

a. Increased Low Frequency Filtering for Ultrasound Imaging by Using a Mean Removal Post-Processing Algorithm

The purpose of wall filtering in standard power Doppler ultrasound equipment is to remove the effect of reflections from blood vessel walls, vessel movements, muscles, tissue, non blood vessel objects, and all non-moving type ultrasound signals that interfere with the creation and detection of the power Doppler signals resulting from motion of blood in the blood vessels.

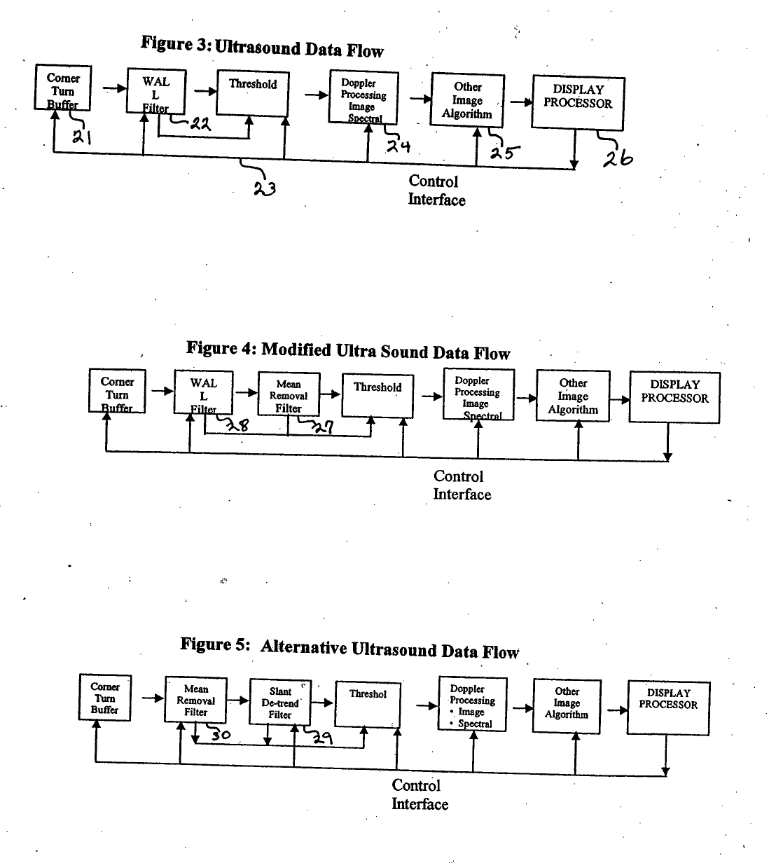

Received Doppler Ultrasound signals after down conversion consists of three complements: BMODE (i.e., unshifted reflections of the transmitted beam), low frequency tissue noise (muscles, moving blood vessels that produce little Doppler shift) and mid to high frequency Doppler signals. The wall filter's purpose is remove the BMODE and low frequency tissue noise while leaving the Doppler signal produced by moving blood for post-processing analysis and display. An exemplary processing signal flow is illustrated in FIG. 3.

In FIG. 3 digital information from the beam former 7 is buffered 21 and passed to a wall filter 22. Signals above a threshold, controlled by the process control 1 through the control interface 23 are processed for image spectral content and further processed 25 to supply the spatial and temporal content needed by the display processor 26.

The wall filter's simple design constraint is to have a very narrow low pass notch that rejects DC and the low frequency signals while passing the higher frequency components. These filtered signals are then post-processed to form imagery. Ultrasound Wall filtering is generally performed over very small number of pulses, which makes the window of observation very short and difficult to post-process reliably without introducing large bias errors or removing too much of the Doppler signal sought after.

One form of the herein-disclosed invention, illustrated schematically in FIG. 4, produces a more robust and simpler wall filtering solution, achieved by cascading a mean removal filter in series after a standard wall filter. In FIG. 4 an additional digital filter 27 calculates the mean value of the signals passing the wall filter 28. This algorithm's advantage over standard wall filtering techniques is increased depth of the filter notch at DC without sacrificing sought after power Doppler signal. This has an advantage in increasing overall sensitivity by increasing Doppler signal content relative to the signal noise.

b. Increased Low Frequency Filtering for Ultrasound Imaging by Using a Combination Mean Removal and De-trending Filtering Algorithm

Another form of the herein-disclosed invention produces a more robust/simpler wall filtering solution can be achieved by removing the wall filter function and replacing it with a cascaded Slant De-trend Filter in series with a Mean Removal Filter. The mean removal algorithm removes the observed pulses' DC components (i.e. BMODE and tissue noise). The de-trending algorithm isolates and removes the low frequency vibrations observed across the pulses processed, which removes the lower frequency signals by subtracting any calculated ramp function. This algorithm's advantage over standard wall filtering techniques is increased depth of the filter notch at DC without sacrificing sought after power Doppler signal. This has an advantage in increasing overall sensitivity by increasing Doppler signal content. This solution is illustrated schematically in FIG. 5, showing a mean removal filter 30 and a trend removal filter 29 in place of the wall filter 22, of FIG. 3.

c. Increased Low Frequency Filtering for Ultrasound Imaging by Cascading Wall Filter, Mean Removal and a Slant De-trending Algorithm

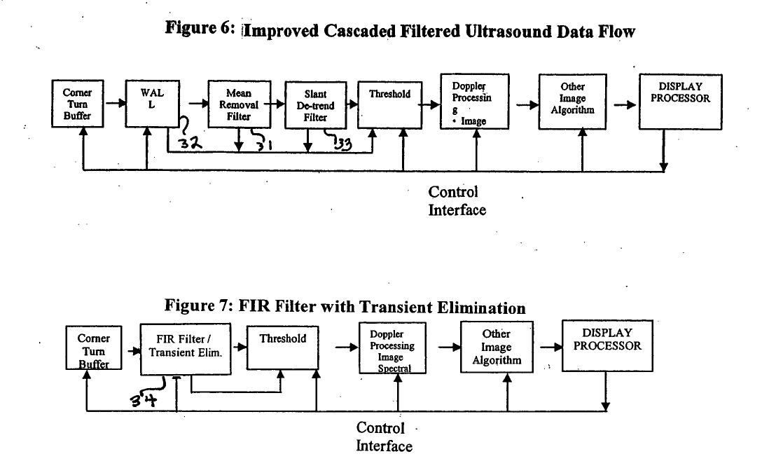

Another form of the herein-disclosed invention produces a more robust and simpler wall filtering solution by cascading a Slant De-trend Filter 33 in series with a Mean Removal Filter 31 and Wall Filter 32. The wall filter removes the majority of the DC components and some of the low frequency signals. The mean removal algorithm removes more DC signal and attenuates additional low frequency components close to DC (i.e. moving vessel walls, tissue noise). The de-trending algorithm further isolates and removes the remaining low frequency vibrations observed across the pulses processed. This algorithm's advantage over standard wall filtering techniques is an increased width depth of the filter notch at DC without sacrificing sought after power Doppler signal. This has an advantage in increasing overall sensitivity by increasing Doppler signal content. This form of the invention is illustrated schematically in FIG. 6.

d. Transient-eliminated Wall Filter

This filtering solution uses a finite impulse response filter whose taps sum to zero and elimination of the transient portions of the filter output.
A finite impulse response (FIR) filter is defined by its response

$$h ( t ) , t = 0 , 1 , 2 , … , N - 1$$

$$to a unit impulse , \\ δ ( t ) = \{ \begin{matrix}1 , t = 0 \\ 0 , t \neq 0\end{matrix}$$

It's response to an input sequence, u(t), of length M, is conventionally given by an output sequence of length M, where each output sample is a weighted sum of previous input samples

$$y ( t ) = {\sum }_{n = 0}^{N - 1} h ( n ) u ( t - n ) , t = 0 , 1 , … M - 1.$$

If h(t) is designed so that its terms sum to zero, one would expect zero output if the input is constant. Unfortunately, his is not true during the start-up transient of the filter, i.e., for t<N. By eliminating the first N output samples we guarantee zero output for a constant input.

This filtering solution is illustrated in FIG. 7, which shows the data stream passing through a finite impulse response filter with transient elimination 34.

It will be understood that the embodiments described herein are merely exemplary and that a person skilled in the art may make many variations and modifications without departing from the spirit and scope of the invention. All such variations and modifications are intended to be included within the scope of the invention.

1. A method for displaying data obtained by transmitting ultrasonic energy into a subject body, which body includes blood flowing in blood vessels bounded by the vessels' walls and supporting tissues having muscles and tissue, comprising the steps of:
receiving reflected ultrasonic energy from the subject body, which reflected energy comprises Doppler shifted signals reflected by the blood and unshifted signals and signals Doppler shifted by less than a threshold amount by the vessels' walls and the supporting tissues; and
digitally processing the reflected ultrasonic energy producing a data stream in order to measure and display resultant data related to the blood flowing in the blood vessels,
wherein the digitally processing step comprises filtering to remove the unshifted signals and the signals Doppler shifted by less than a threshold amount by digitally processing the data stream sequentially in a wall filter and a mean removal filter.

2. A method of claim 1 comprising processing the resultant data to produce recordable or displayable information related to the blood flowing in the blood vessels.

3. A method of claim 2 comprising recording or displaying the information related to the blood flowing in the blood vessels.

4. A method of claim 2 wherein the information related to the blood flowing in the blood vessels comprises vector velocity and flow volume.

5. A method for displaying data obtained by transmitting ultrasonic energy into a subject body, which body includes blood flowing in blood vessels bounded by the vessels' walls and supporting tissues having muscles and tissue, comprising the steps of:
receiving reflected ultrasonic energy from the subject body, which reflected energy comprises Doppler shifted signals reflected by the blood and unshifted signals and signals Doppler shifted by less than a threshold amount by the vessels' walls and the supporting tissues; and
digitally processing the reflected ultrasonic energy producing a data stream in order to measure and display resultant data related to the blood flowing in the blood vessels,
wherein the digitally processing step comprises filtering to remove the unshifted signals and the signals Doppler shifted by less than a threshold amount by digitally processing the data stream sequentially in a mean removal filter and a trend removal filter.

6. A method of claim 5 comprising processing the resultant data to produce recordable or displayable information related to the blood flowing in the blood vessels.

7. A method of claim 6 comprising recording or displaying the information related to the blood flowing in the blood vessels.

8. A method of claim 6 wherein the information related to the blood flowing in the blood vessels comprises vector velocity and flow volume.

9. A method for displaying data obtained by transmitting ultrasonic energy into a subject body, which body includes blood flowing in blood vessels bounded by the vessels' walls and supporting tissues having muscles and tissue, comprising the steps of:
receiving reflected ultrasonic energy from the subject body, which reflected energy comprises Doppler shifted signals reflected by the blood and unshifted signals and signals Doppler shifted by less than a threshold amount by the vessels' walls and the supporting tissues; and
digitally processing the reflected ultrasonic energy producing a data stream in order to measure and display resultant data related to the blood flowing in the blood vessels,
wherein the digitally processing step comprises filtering to remove the unshifted signals and the signals Doppler shifted by less than a threshold amount by digitally processing the data stream sequentially a wall filter, a mean removal filter and a trend removal filter.

10. A method of claim 9 comprising processing the resultant data to produce recordable or displayable information related to the blood flowing in the blood vessels.

11. A method of claim 10 comprising recording or displaying the information related to the blood flowing in the blood vessels.

12. A method of claim 10 wherein the information related to the blood flowing in the blood vessels comprises vector velocity and flow volume.

13. A method for displaying data obtained by transmitting ultrasonic energy into a subject body, which body includes blood flowing in blood vessels bounded by the vessels' walls and supporting tissues having muscles and tissue, comprising the steps of:
receiving reflected ultrasonic energy from the subject body, which reflected energy comprises Doppler shifted signals reflected by the blood and unshifted signals and signals Doppler shifted by less than a threshold amount by the vessels' walls and the supporting tissues; and
digitally processing the reflected ultrasonic energy producing a data stream in order to measure and display resultant data related to the blood flowing in the blood vessels,
wherein the digitally processing step comprises filtering to remove the unshifted signals and the signals Doppler shifted by less than a threshold amount by digitally processing the data stream in a finite impulse response filter with transient elimination.

14. A method of claim 13 comprising processing the resultant data to produce recordable or displayable information related to the blood flowing in the blood vessels.

15. A method of claim 14 comprising recording or displaying the information related to the blood flowing in the blood vessels.

16. A method of claim 14 wherein the information related to the blood flowing in the blood vessels comprises vector velocity and flow volume.

17. A device for digitally processing a data stream resulting from detection of ultrasound reflections from a subject including blood flowing within blood vessel walls, in order to remove unshifted signals and signals Doppler shifted by less than a threshold amount, the device comprising: a wall filter, a mean removal filter, and a threshold module which are sequentially connected.

18. A device of claim 17 comprising a Doppler processing algorithm to produce image data and spectral data.

19. A device of claim 18 comprising an image algorithm to supply spatial and temporal data required for storage or display.

20. A device for digitally processing a data stream resulting from detection of ultrasound reflections from a subject including blood flowing within blood vessel walls, in order to remove unshifted signals and signals Doppler shifted by less than a threshold amount, the device comprising: a mean removal filter and a trend removal filter connected sequentially.

21. A device of claim 20 comprising a Doppler processing algorithm to produce image data and spectral data.

22. A device of claim 21 comprising an image algorithm to supply spatial and temporal data required for storage or display.

23. A device for digitally processing a data stream resulting from detection of ultrasound reflections from a subject including blood flowing within blood vessel walls, in order to remove unshifted signals and signals Doppler shifted by less than a threshold amount, the device comprising: a wall filter, a mean removal filter, and a trend removal filter which are sequentially connected.

24. A device of claim 23 comprising a Doppler processing algorithm to produce image data and spectral data.

25. A device of claim 24 comprising an image algorithm to supply spatial and temporal data required for storage or display.

26. A device for digitally processing a data stream resulting from detection of ultrasound reflections from a subject including blood flowing within blood vessel walls, in order to remove unshifted signals and signals Doppler shifted by less than a threshold amount, the device comprising: a finite impulse response filter with transient elimination.

27. A device of claim 26 comprising a Doppler processing algorithm to produce image data and spectral data.

28. A device of claim 27 comprising an image algorithm to supply spatial and temporal data required for storage or display.
